(19) United States
(12) Patent Application Publication
(10) Pub. No.: US 2022/0273271 A1
(43) Pub. Date: Sep. 1, 2022

(54) MEDICAL FILTRATION SYSTEM

(71) Applicant: Control Medical Technology, LLC, Hallandale, FL (US)

(72) Inventor: Shawn P. Fojtik, Park City, UT (US)

(21) Appl. No.: 17/683,270

(22) Filed: Feb. 28, 2022

Related U.S. Application Data
(60) Provisional application No. 63/154,704, filed on Feb. 27, 2021.

(51) Int. Cl. A61B 10/00; B01D 46/24; B01D 46/42

(52) U.S. Cl. CPC A61B 10/0045; B01D 46/2403; B01D 46/4254; B01D 2275/201

(57) ABSTRACT

A filtration system may include a chamber and a filter disposed within an interior the chamber. A closed end, or a tip, of the filter may face an inlet into the interior of the chamber. An open end, or a base, of the filter may surround an outlet from the interior of the chamber. As a sample passes through the interior of the chamber, solid and/or semisolid material in the sample may be collected between an exterior surface of the filter and an interior surface of the chamber, and may be viewed through the chamber.

### CROSS-REFERENCE TO RELATED APPLICATION

A claim for priority to the Feb. 27, 2021 filing date of U.S. Provisional Patent Application No. 63/154,704, titled MEDICAL FILTRATION SYSTEM AND USE OF THE SAME DURING ASPIRATION (“the '704 Provisional Application”) is hereby made pursuant to 35 U.S.C. § 119(e). The entire disclosure of the '704 Provisional Application is hereby incorporated herein.

### TECHNICAL FIELD

This disclosure relates to systems used to obtain samples from a body of a subject and, more specifically, to medical filtration systems that facilitate sample collection. This disclosure also relates to aspiration systems and to aspiration processes.

### RELATED ART

Catheters, sheaths, tubing, trocars, wires, and other devices may be used with syringes, hand-pump aspirators, electric pumps, and wall suction to remove a variety of fluids, pathology, embolics, and tissue, including thrombi, from numerous vessels, cavities, chambers, appendages, anomalies, and other part of a body. Examples of procedures include, but are not limited to: aspiration of biopsy samples, cysts, thrombi, foreign bodies, polyps (e.g., following polypectomy, etc.), and biliary abscesses; nephrostomy; plural effusion; clearing and cleaning tubes and catheters; spinal disc decompression; live tissue resection; lavage; sinus, cavity, and wound cleaning; cancer diagnostics and therapies; and general drainage, to name only a few. Existing aspiration devices can be ineffective, difficult to use, create complications, and suffer from a variety of other deficiencies.

### SUMMARY

A filtration system according to this disclosure may at least momentarily capture solid and/or semisolid material in a sample as it is aspirated from a body of a patient, which may enable a clinician (an individual) to conduct a real-time visual analysis of the sample as it is aspirated from the body of the patient. Thus, the filtration system may also enable the clinician to determine whether an elongated medical device (e.g., an aspiration catheter, tubing, a sheath, a cannula, a trocar, a needle, a wire, etc.) is properly positioned within the body of the subject.

The filtration system may include a chamber that may be disposed proximal to (i.e., away from the patient, toward a clinician side of) an elongated medical device. A filter is disposed within an interior the chamber. A closed end, or a tip, of the filter may face an inlet into the interior of the chamber. An open end, or a base, of the filter may surround an outlet from the interior of the chamber. A sample (e.g., fluid, tissue, etc.) that flows into the interior of the chamber must pass through the filter before the sample exits the interior of the chamber. A pump may be associated with the chamber in a manner that draws the sample from the body of the patient, through the elongated medical device, into the interior of the chamber, onto and partially through the filter. An arrangement of the filter within the chamber may enable the sample to be captured between an exterior surface, or outside, of the filter and an interior surface of a wall of the chamber as a sample is drawn proximally into and through the chamber.

The chamber may be transparent. Transparency of the chamber may enable a clinician to visualize its contents as they are aspirated into the chamber and captured by the filter. A wall of the chamber may be flexible. Flexibility of the wall of the chamber may render it compressible and, thus, squeezable. A configuration of the chamber may enable it to be opened to provide access to its contents (e.g., a sample that has been aspirated from the patient's body and captured by the filter, etc.).

A shape and dimensions of a cross-section of the chamber, taken transverse to a length of the chamber, may be the same or substantially the same (e.g., accounting for manufacturing tolerances, etc.) along the length of the chamber. As an example, the chamber may be cylindrical in shape. In other examples, the chamber may have the shape of a polygonal prism.

The dimensions of the chamber may impart its interior with a relatively small volume. A volume of the interior of the chamber may be tailored to prevent fogging of, or the condensation of moisture on, surfaces of the chamber as body temperature samples from the body of the patient are introduced into a cooler, ambient (e.g., room temperature, etc.) environment.

Dimensions of a cross-section of the filter taken transverse to the length of the filter may increase from a distal side of the filter to a proximal side of the filter; thus, the filter may taper outwardly from a location along its distal side toward a location along its proximal side. Such a filter may be conical or somewhat conical in shape (e.g., a truncated cone shape, or a frustoconical shape; a pyramidal shape; a truncated pyramidal shape; etc.).

As the filter is designed to be disposed within an interior of the chamber, a maximum dimension (e.g., an outer diameter, etc.) across a cross-section transverse to the length of the filter may be the same or less than a dimension across the interior of the chamber (e.g., an inner diameter etc.). As an example, in embodiments where the filter has a somewhat conical shape, a dimension across the open end, or the base, of the filter may be about the same or slightly smaller than a dimension across the interior of the chamber, enabling the filter to completely cover the outlet from the interior of the chamber.

A shape of a cross-section of the filter, taken transverse to a length of the filter, may be the same or substantially the same along the length of the filter. The cross-sectional shape of the filter may be the same as the cross-sectional shape of the chamber. For example, in embodiments where the chamber is cylindrical in shape, the filter may be conical or frustoconical in shape. As another example, in embodiments where the chamber has the shape of a polygonal prism (e.g., a triangular prism, a square prism, a regular pentagonal prism, a regular hexagonal prism, etc.), the filter may have the shape of a pyramid or truncated pyramid with a base having the same polygonal shape (e.g., triangular, square, regular pentagonal, regular hexagonal, etc.) as the polygonal prism.

Alternatively, a shape and dimensions of a cross-section of the filter, taken transverse to the length of the filter, may both be the same or substantially the same along the length of the filter. Such a filter may completely cover an outlet of the chamber. The cross-sectional dimensions of such a filter may be smaller than corresponding dimensions of the interior of the chamber, spacing exterior surfaces of at least a portion of the filter apart from interior surfaces of walls of the chamber.

In some embodiments, a length of the interior of the chamber may exceed a length of the filter. Thus, the filter may be slide along the length of the interior of the chamber. In embodiments where the filter is able to slide along the length of the interior of the chamber, a spring may be associated with the filter to urge, or bias, it toward one end of the interior of the chamber. Without limitation, a spring may urge the filter toward a distal end of the interior of the chamber (i.e., toward the patient) while enabling the filter to slide proximally as a sample is drawn proximally into and through the interior of the chamber.

A sample aspiration system may include an elongated medical device, the filtration system, and an aspirator. The elongated medical device may be introducible into a body of a patient, with a distal end of the elongated medical device being positionable at a location from which a sample is to be obtained from the body. The filtration system may communicate with a proximal end of the elongated medical device. The aspirator may be positioned on a proximal side of the filtration system to apply a vacuum to the filtration system and to the elongated medical device and, thus, to drawn the sample from the body of the patient. A sample aspiration may also include other components, such as one or more flow control switches, tissue resectors, tubes, branches, valves, and the like.

A sample aspiration method according to this disclosure may include positioning a distal end of an elongated medical device at a location within the body of the patient from which the sample is to be obtained. A proximal end of the elongated medical device may communicate with an inlet of a chamber of a filtration system. In addition, an aspirator may be placed in fluid communication with an outlet of the chamber. With the distal end of the elongated medical device in place, the aspirator may be used to draw a sample from within the body of the patient into and through the elongated medical device and into and through the chamber.

As the sample is drawn into and through the chamber, solid and/or semisolid material within the sample may be captured between an exterior surface of a filter of the filtration system, with the filter being located within an interior of the chamber and an interior surface of a wall of the chamber. More specifically, the chamber and filter may be oriented such that a closed end of the filter is oriented distally while an open end of the filter is oriented proximally and covers an outlet of the chamber. Thus, a sample that flows into the interior of the chamber will first encounter the closed end of the filter and must encounter the filter before a portion of the sample exits the interior of the chamber through the outlet.

The sample may be viewed as the sample enters into and passes through the interior of the chamber. Portions of the sample (e.g., solid portions of the sample, semisolid portions of the sample, etc.) that are trapped by the filter may also be viewed. Viewing of the sample may occur through the wall of the chamber.

In some embodiments, a clinician (an individual) may hold the chamber as a sample is aspirated into and passes through the chamber. In embodiments where the walls of the chamber are flexible, the clinician may feel, or non-visibly sense, movement of the filter along a length of the interior of the chamber. In addition, the clinician may squeeze the chamber. By squeezing of the chamber, the clinician may facilitate the flow of the sample through the filter, force solid and/or semisolid material of the sample through the filter, or otherwise affect the sample collection process.

While a volume within an interior of the chamber may be tailored to minimize condensation, or fogging, from occurring as a relatively warm sample is drawn into a relatively cool environment, any fogging that occurs as a sample travels through the chamber may be reduced or eliminated, or the chamber may be defogged, by tilting or turning the chamber as at least a portion of a sample travels therethrough.

If the solid and/or semisolid material captured by the filter impedes sample collection, the filtration system and the portion of the sample collected thereby may be replaced with a fresh filtration system. More specifically, the chamber may be disconnected from between the elongated medical device and from the aspirator and a chamber of another, fresh, filtration system may be coupled between the elongated medical device and the aspirator. Any portions of the sample that remain within the interior of the chamber (e.g., portions of the sample that have been captured between the exterior surface of the filter and the interior surface of a wall of the chamber, etc.) may be removed from the chamber.

A filtration system, a sample collection system, and method of collecting a sample according to this disclosure may enable solid and/or semisolid materials (e.g., embolics, clots, thrombi, pathology, etc.) to be aspirated, or pulled, from the body of a patient in such a way that the solid and/or semisolid materials may be trapped between an exterior surface of the filter and a transparent wall of the chamber. Such an arrangement enables clinicians to physically see the solid and/or semisolid materials once they emerge from the patient's body. If the solid and/or semisolid materials were instead trapped within the interior of the filter, as would occur if the filter were oriented with its larger base oriented distally and its smaller tip oriented proximally, it would otherwise be difficult for clinicians to physically visualize the solid and/or semisolid materials, as the filter and potentially translucent or opaque fluids (e.g., blood, etc.) between the exterior surface of the filter and the wall of the container would block the clinicians' views.

Other aspects of this disclosure, as well as features and advantages of various aspects of this disclosure, should become apparent to those of ordinary skill in the art through consideration of the ensuing description, the accompanying drawings, and the appended claims.

### BRIEF DESCRIPTION OF THE DRAWINGS

In the drawings:

FIG. 1 is a side view of an embodiment of a filtration system according to this disclosure;

FIGS. 2 and 3 show an embodiment of a sample collection system in which the filtration system is located between a proximal side of an elongated medical device and a distal side of an aspiration tube;

FIG. 4 shows an embodiment of a sample collection system in which a sample passes through a tissue resector, or macerator, before it enters into the filtration system; and

FIGS. 5-7 show an embodiment of a method for collecting a sample in which the sample is filtered in a manner that prevents blockage while facilitating the collection of solid and, optionally, semisolid portions of the sample.

### DETAILED DESCRIPTION

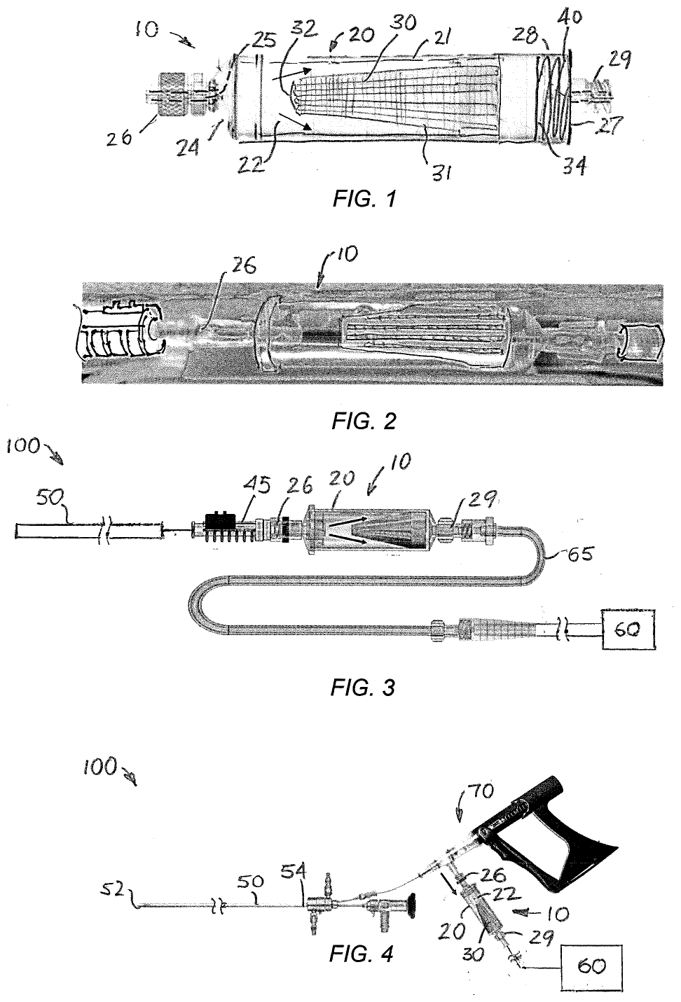

As illustrated by FIG. 1, a filtration system 10 includes a chamber 20 and a filter 30 within an interior 22 of the chamber 20. The chamber 20 includes a distal side 24 and a proximal side 27. An inlet 25 on the distal side 24 of the chamber 20 may enable fluid (e.g., a sample, etc.) to flow into the interior 22 of the chamber 20. An outlet 28 on the proximal side 27 of the chamber 20 may enable fluid to flow out of the interior 22 of the chamber 20. A distal coupling element 26 (e.g., a luer lock connector, etc.) may be provided on the distal side 24 of the chamber 20. A proximal coupling element 29 (e.g., a luer lock connector, a stepped connector, etc.) may be provided on the proximal side 27 of the chamber 20.

The chamber 20 and its interior 22 may be cylindrical in shape. The interior 22 of the chamber 20 may have an inner diameter (ID) as large as or larger than the inner diameter of an elongated medical device 50 (e.g., a catheter, tubing, a trocar, a wire, etc.) with which the filtration system 10 is to be used. The inner diameter of the interior 22 may be as large as or larger than the maximum inner diameter of an elongated medical device 50 (FIGS. 3 and 4) that may be used to aspirate fluid from the body of an individual. In a specific embodiment, the chamber 20 may have an inner diameter of about 14 mm to about 18 mm. An outer diameter (OD) of such a chamber 20 may be about 16 mm to about 20 mm. A thickness of a wall 21 of the chamber 20 may be about 1 mm, about 0.5 mm, about 0.25 mm, or less. The chamber 20 may have a length of about 10 mm to about 120 mm (e.g., about 60 mm, etc.). Chambers 20 with greater lengths may be desired in certain applications to allow more filtrate (e.g., by volume, etc.) to accumulate in the interior 22 of the chamber 20.

At least a portion of the wall 21 of the chamber 20 may be transparent. The chamber 20 may be formed from any of a variety of suitable materials. Suitable materials for manufacturing the chamber 20 may include, without limitation, rigid or flexible polyurethane, polypropylene, polyethylene, silicone, and/or any other suitable polymer or polymer blend. The chamber 20 may be molded, extruded, additive-manufactured (e.g., 3D printed, etc.), or formed by any of a variety of other suitable manufacturing techniques.

In some embodiments, the wall 21 of the chamber 20 may be flexible. A material from which the wall 21 of the chamber 20 is formed and/or a thickness of the wall 21 may render the wall 21 flexible. A flexibility of the wall 21 may enable a clinician (an individual) to compress the wall 21, which may enable the clinician to feel the contents of the interior 22 of the chamber 20 (e.g., the filter 30, a sample, etc.) and/or squeeze the chamber 20 in a manner that facilitates flow of the sample through the chamber 20.

The filter 30 of the filtration system 10 may comprise an elongated filter. The filter 30 may comprise a submersible filter. The filter 30 may be positioned within the interior 22 of the chamber 21. In some embodiments, such as that depicted by FIG. 1, a closed end 32 of the filter 30 may be smaller than an open end 34 of the filter 30. A wall 31 of the filter may taper outwardly from the closed end 32 to the open end 34. Thus, the filter may be somewhat conical in shape (e.g., it may be conical, frustoconical, pyramidal, have the shape of a truncated pyramid, etc.). The closed end 32 of such a filter 30 may be oriented toward the inlet 25 to the interior 22 of the chamber 20 (i.e., distally) and the open end 34 of such a filter 30 may be oriented toward the outlet 28 from the interior 22 of the chamber 20 (i.e., proximally).

A length of the filter 30 is, of course, the same as or less than a length of the interior 22 of the chamber 20. In addition, the outer diameter of the open end 34 of the filter 30 may be the same as or slightly smaller than the inner diameter of the interior 22 of the chamber 20 to enable the filter 30 to be positioned within the interior 22 of the chamber 20. In some embodiments, the filter 30 may have a length of up to about 100 mm. In a more specific embodiment, the filter 30 may have a length of about 40 mm. An outer diameter of the closed end 32, or the tip or distal end, of such a filter 30 may be about 6 mm to about 10 mm and an outer diameter of the open end 34, or the base or proximal end, of such a filter may be about 17 mm to about 18 mm.

In embodiments where a length of the filter 30 is shorter than a length of the interior 22 of the chamber 20, the filter 30 may be able to slide within the interior 22 of the chamber 20. In such embodiments, the filter 30 may initially be positioned distally, toward the inlet 25 to the interior 22 of the chamber 20. As fluids are drawn into and through the interior 22 of the chamber 20, along with any solid and/or semisolid materials, the filter 30 may move proximally as the filter 30 traps the solid and/or semisolid materials within the interior 22 of the chamber 20. Such movement of the filter 30 within the interior 22 of the chamber 20 may be audibly discernable to clinicians (i.e., they may hear it). In embodiments where the chamber 20 has thin, flexible walls, clinicians may also feel movement of the filter 30 within the interior 22 of the chamber 20. One or both of these aspects may enable clinicians to sense and determine when solid and/or semisolid materials are present (or at least likely to be present) within the interior 22 of the chamber 20 without looking at the chamber 20, which might require the clinician to take his or her eyes off the surgical access and/or fluoroscopic monitoring screens/monitors. Once the clinician receives an indication that solid or semisolid material has been trapped by the filter 30, he or she may visualize the solid or semisolid material through the wall 21 of the chamber 20.

In embodiments where the filter 30 is shorter than the interior 22 of the chamber 20, the filtration system 10 may include a spring 40. The spring 40 may comprise a compression spring positioned between the filter 30 and a proximal side of the interior 22 of the chamber 20. The spring 40 may enable the filter 30 to move proximally as the filter 30 traps solid and/or semisolid material within the interior 22 of the chamber 20 but force the filter 30 distally when solid and/or semisolid materials are cleared from the interior 22 of the chamber 20; for example, as the solid and/or semisolid materials are forced through the filter 30 (e.g., by way of normal fluid dynamics, as the clinician squeezes the chamber, etc.) or are otherwise removed from the interior 22 of the chamber 20.

The wall 31 of the filter 30, including the closed end 32 of the filter 30, may include apertures. The apertures may have shapes and sizes that enable a sample to pass through the wall 31 of the filter 30 while at least momentarily capturing solid and/or semisolid particles of the sample that have at least one dimension that exceeds a dimension across the apertures. The apertures may have any shape (e.g., slits, pores or other openings, etc.) and arrangement suitable for the type of sample to be obtained from the body of the patient. For example, the apertures may comprise vertical (i.e., oriented along the length of the filter 30) slits, horizontal (i.e., oriented around the circumference of the filter 30) slits, off-axis angled slits, round holes, vertical oval holes, horizontal oval holes, off-axis oval holes, and/or any combination of slits and/or holes, as well as any of or any combination of different sizes of apertures.

The sizes, shapes, and number of apertures through the wall 31 of the filter 30 may minimize interruption to or disturbance of the flow of fluid (e.g., liquid, gas, etc.) (e.g., reduction in flow rate, introduce additional turbulence into the fluid flow, etc.) through the interior 22 of the chamber 20 and the filter 30 therein. As shown in the table that follows, per Poisuille's laws on fluid dynamics and laminar flow through a tube, all other things being equal, if the diameter of a tube is reduced 50%, flow is decreased to about one-sixteenth ( 1/16), or by about 16 times.

TABLE

| Diameter | Radius | Pressure Δ | Viscosity | Length | Flow |
|---|---|---|---|---|---|
| mm | mm | mm/Hg | CP | mm | ml/sec |
| 0.25 | 0.125 | 500 | 1 | 1 | 0.006 |
| 0.5 | 0.25 | 500 | 1 | 1 | 0.102 |
| 0.75 | 0.375 | 500 | 1 | 1 | 0.518 |
| 1 | 0.5 | 500 | 1 | 1 | 1.636 |
| 1.25 | 0.625 | 500 | 1 | 1 | 3.99 |
| 1.5 | 0.75 | 500 | 1 | 1 | 8.28 |
| 2 | 1 | 500 | 1 | 1 | 26.18 |
| 2.25 | 1.125 | 500 | 1 | 1 | 41.94 |
| 2.5 | 1.25 | 500 | 1 | 1 | 63.92 |
| 2.75 | 1.375 | 500 | 1 | 1 | 93.58 |
| 3 | 1.5 | 500 | 1 | 1 | 132.54 |

Accordingly, if laminar suction is applied to the outlet 28 from the interior 22 of the chamber 20 through a tube (not shown), with both the outlet 28 and the tube having inner diameters of 3 mm and the filter 30 includes at least 256 circular apertures with diameters of 0.75 mm (132.54 ml/s/0.518 ml/s=255.9), then the filter 30 will not disrupt the suction applied to the filtration system 10 and the sample collection system of which the filtration system 10 is a part. Conversely, such a filter would not disrupt a laminar flow of a fluid with a viscosity of 1 cp or less that lacks solid or semisolid material.

The filter 30 may be formed from any suitable material. Examples of suitable materials include, but are not limited to rigid or flexible metals, rigid or flexible polymers (e.g., polyurethane, polypropylene, polyethylene, silicone, etc.) or polymer blends, or the like. A metal filter may be stamped, etched, and/or cut. A polymer filter 30 may be molded, extruded, additive-manufactured, or formed by any other suitable manufacturing process.

With reference to FIGS. 2 and 3, the filtration system 10 may be coupled to a flow control switch 45, which may provide for selective control over whether the filtration system 10 is in flow communication with an elongated medical device 50. More specifically, the flow control switch 45 may be removably coupled to the distal coupling element 26 of the chamber 20 of the filtration system 10. Optionally, the flow control switch 45 may be omitted from a sample collection system according to this disclosure.

FIG. 3 depicts an embodiment of a sample collection system 100 that includes the filtration system 10, an elongated medical device 50, and an aspirator 60. The sample collection system 100 may also include other non-essential components, such as the flow control switch 45 between the elongated medical device 50 and the filtration system, tubing 65 that establishes communication between the interior of the chamber 20 of the filtration system 10 and the aspirator 60, and/or other components, as illustrated by FIG. 3.

The filtration system 10 may be coupled directly or indirectly (e.g., by way of one or more intervening elements, such as the illustrated flow control switch 45, etc.) to the elongated medical device 50 by way of the distal coupling element 26. The elongated medical device 50 may comprise any suitable device for obtaining a sample from the body of the patient. Some non-limiting examples of elongated medical devices 50 include aspiration catheters, tubing, sheaths, cannulas, trocars, needles, wires, and the like.

The filtration system 10 may also be coupled directly or indirectly (e.g., by way of tubing 65, etc.) to the aspirator 60. More specifically, the proximal coupling element 29 of the chamber 20 of the filtration system 10 may directly or indirectly couple the filtration system 10 to the aspirator 60. The aspirator 60 may comprise any suitable pump or other aspiration device. As a non-limiting example, the aspirator 60 may comprise an aspiration device such as that described by U.S. Pat. No. 8,491,539, the entire disclosure of which is hereby incorporated herein.

Another embodiment of sample collection system 100′ according to this disclosure is depicted by FIG. 4. The sample collection system 100′ includes an elongated medical device 50, a tissue resector 70 that receives a sample from the elongated medical device 50, the filtration system 10, and an aspirator 60. Such a sample collection system 100′ may also include one or more other non-essential components, such as those shown in FIG. 4.

The elongated medical device 50 may comprise any suitable device for obtaining a sample from the body of the patient. Some non-limiting examples of elongated medical devices 50 include aspiration catheters, tubing, sheaths, cannulas, trocars, needles, wires, and the like.

The tissue resector 70, which is positioned proximal to the elongated medical device 50, may mechanically break up, cut, or macerate, any solid and/or semisolid materials present in a sample obtained with the elongated medical device 50. As a non-limiting example, the tissue resector 70 may comprise a device of the type described by U.S. Pat. No. 10,667,836, the entire disclosure of which is hereby incorporated herein. By macerating a sample, the tissue resector 70 may reduce the sizes of solid and/or semisolid materials within the sample to enable them to flow through the filter 30 of the filtration system 10.

The filtration system 10 may be coupled directly or indirectly (e.g., by way of one or more intervening elements, to the tissue resector 70 by way of the distal coupling element 26 of the chamber 20.

The filtration system 10 may also be coupled directly or indirectly (e.g., by way of tubing 65, etc.) to the aspirator 60. More specifically, the proximal coupling element 29 of the chamber 20 of the filtration system 10 may directly or indirectly couple the filtration system 10 to the aspirator 60. The aspirator 60 may comprise any suitable pump or other aspiration device. As a non-limiting example, the aspirator 60 may comprise an aspiration device such as that described by U.S. Pat. No. 8,491,539.

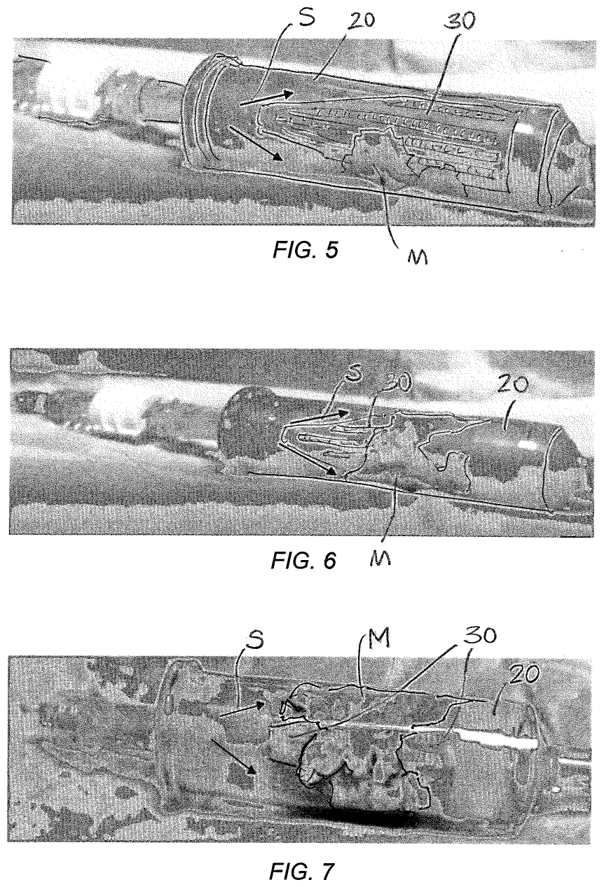

With continued reference to FIG. 4 and with reference to FIGS. 5-7, a method for aspirating and a sample S may include positioning a distal end 52 of an elongated medical device 50 at a location within the body of the patient from which the sample S is to be obtained. A proximal end 54 of the elongated medical device 50 may communicate with an inlet 25 (FIG. 1) of the chamber 20 of the filtration system 10. In addition, an aspirator 60 may be placed in fluid communication with an outlet 28 (FIG. 1) of the chamber 20. With the distal end 54 of the elongated medical device 50 in place, the aspirator 60 may be used to draw a sample S from within the body of the patient into and through the elongated medical device 50 and into and through the chamber 20.

As the sample S is collected and travels through the interior 22 of the chamber 20, an orientation of the filter 30 within interior 22 of the chamber 20 may capture solid and/or semisolid material M between an exterior surface of the filter 30 and an interior surface of the chamber 10. More specifically, the chamber 20 and filter 30 may be oriented such that a closed end 32 (FIG. 1) of the filter 30 is oriented distally while an open end 34 (FIG. 1) of the filter 30 is oriented proximally and covers an outlet 28 of the chamber 20. Thus, a sample S that flows into the interior 22 of the chamber 20 will first encounter the closed end 32 of the filter 30 and must encounter the filter 30 before a portion of the sample S exits the interior 22 of the chamber 20 through the outlet 28.

The sample S, including any captured solid and/or semisolid material M, may be viewed as the sample S enters into and passes through the interior 22 of the chamber 20. Solid and/or semisolid material M of the sample S that are trapped by the filter 30 may also be viewed. Viewing of the sample S and any solid and/or semisolid material M may occur through the wall of the chamber. FIGS. 5-7 depict the progressive collection of solid and/or semisolid material M between the exterior surface of the filter 30 and the interior surface of the chamber 20 as a sample S flows into and through the interior 22 of the chamber 20. In some embodiments, aspiration may be reduced or at least temporarily terminated while the clinician views the solid and/or semisolid material M.

In some embodiments, a clinician (an individual) may hold the chamber 20 as a sample S is aspirated into and passes through the interior 22 of the chamber 20. In embodiments where the walls 21 (FIG. 1) of the chamber 20 are flexible, the clinician may feel, or non-visibly sense, movement of the filter 30 along a length of the interior 22 of the chamber 20, which may indicate that solid and/or semisolid material M is being trapped between the exterior surface of the filter 30 and the interior surface of the chamber 20. In embodiments where a clinician may feel or hear movement of the filter 30 with the interior 22 of the chamber 20, the clinician may watch the surgical access site and/or fluoroscopic monitoring screens and/or monitors until the solid and/or semisolid material M of the sample enters the interior 22 of the chamber 20. Once the clinician receives an indication that solid or semisolid material has been trapped by the filter 30, he or she may quickly visualize the solid or semisolid material through the wall 21 of the chamber 20, then redirect his or her attention to the surgical access site and/or screens or monitors.

The clinician may squeeze the wall(s) 21 (FIG. 1) of the chamber 20. For example, the clinician may compress the chamber by squeezing it with his or her hand (e.g., between his or her thumb and index finger, etc.) before, during, and/or after the chamber fills with fluid (e.g., liquid, gas, and/or any other material; embolic, and/or pathology from the body, etc.). By squeezing the wall(s) 21 of the chamber 20, the clinician may facilitate the flow of the sample S, including the solid and/or semisolid material M thereof, through the filter 30, force the solid and/or semisolid material M of the sample S through the filter 30, or otherwise affect the sample S collection process. Squeezing the chamber 20 may allow the clinician to physically move the sample S around the interior 22 of the chamber 20 and to visualize the sample 20 and any other material M (e.g., embolic, pathology, etc.) removed from the body. Squeezing the chamber 20 may also allow the clinician to agitate, macerate, and manually help move the sample S, including the solid and/or semisolid material M thereof (e.g., embolic, pathology, etc.) through the interior 22 of the chamber 20 and the filter 30 to prevent or clear clogging.

While a volume within the interior 22 of the chamber 20 may be tailored to minimize condensation, or fogging, from occurring as a relatively warm sample is drawn into a relatively cool environment, any fogging that occurs on interior surfaces of the chamber 20 as a sample S travels through the chamber 20 may be reduced or eliminated, or the chamber 20 may be defogged, by tilting or turning the chamber 20 as at least a portion of a sample S travels through the interior thereof.

If the solid and/or semisolid material M captured by the filter 30 impedes collection of a sample S, the filtration system 10 and the solid and/or semisolid material M collected thereby may be replaced with a fresh filtration system 10. More specifically, the chamber 20 may be disconnected from between the elongated medical device 50 and from the aspirator 60 and a chamber 20 of another, fresh, filtration system 10 may be coupled between the elongated medical device 50 and the aspirator 60. Aspiration may then resume. Such a process may be repeated until the aspiration procedure is complete.

Any portions of the sample that remain within the interior 22 of the chamber 20 (e.g., solid and/or semisolid material M captured between the exterior surface of the filter 30 and the interior surface of the chamber 20, etc.) may be saved for later analysis (e.g., for further viewing, for pathology, etc.), at which time they may be removed from the chamber 20.

Although the preceding disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A filtration system that filters medical aspirant obtained from a body of a subject, comprising:
a chamber with a distal side including an inlet port, a chamber interior in communication with the inlet port, and a proximal side including an outlet port in communication with the chamber interior; and
a filter within the chamber interior, the filter being elongated and including an open end, a closed end, and a filter interior between the open end and the closed end, the closed end having a smaller dimension than the open end, the closed end being oriented toward the distal side of the chamber, the open end being oriented toward the proximal side of the chamber.

2. The filtration system of claim 1, wherein the chamber interior is cylindrical in shape and the filter is conical or frustoconical in shape.

3. The filtration system of claim 1, wherein a wall of the chamber is transparent.

4. The filtration system of claim 1, wherein a wall of the chamber is flexible.

5. The filtration system of claim 1, wherein the filter can slide within the chamber interior, along a length of the chamber interior.

6. The filtration system of claim 5, further comprising:
a spring within the chamber interior.

7. The filtration system of claim 6, wherein the spring comprises a compression spring within the chamber interior, between the filter and the proximal side of the chamber.

8. The filtration system of claim 1, further comprising:
an aspirator; and
a tube that establishes communication between the aspirator and the outlet of the chamber.

9. The filtration system of claim 8, wherein apertures of the filter minimize interruption to flow of fluid through the filtration system.

10. The filtration system of claim 8, wherein apertures of the filter enable fluid to flow through the filter at a same rate as the fluid can flow through the tube.

11. The filtration system of claim 8, further comprising:
a catheter that establishes communication between the body of the subject and the inlet of the chamber.

12. A method for aspirating a sample from a body of a subject, comprising:
positioning a distal end of an elongated medical device at a location of the body of the subject from which the sample is to be obtained;
securing the elongated medical device to an inlet of a chamber of a filtration system;
placing an aspirator in fluid communication with an outlet of the chamber;
using the aspirator to draw the sample from the elongated medical device into the chamber; and
capturing solid and/or semisolid material between a filter in an interior of the chamber and a wall of the chamber.

13. The method of claim 12, wherein capturing the solid and/or semisolid material includes capturing the solid and/or semisolid material between a cylindrical wall of the chamber and a side of an elongated filter with a distal end that is smaller than a proximal end of the elongated filter.

14. The method of claim 12, further comprising:
viewing the solid and/or semisolid material through the wall of the chamber.

15. The method of claim 12, further comprising:
manually holding the chamber.

16. The method of claim 15, wherein manually holding the chamber includes sensing movement of the filter along a length of the interior of the chamber.

17. The method of claim 15, wherein manually holding the chamber includes squeezing the chamber to force the solid and/or semisolid material through the filter.

18. The method of claim 12, further comprising:
non-visibly sensing movement of a filter along a length of the interior of the chamber.

19. The method of claim 12, further comprising, after capturing the solid and/or semisolid material:
removing an assembly of the chamber and the filter containing the solid and/or semisolid material from between the elongated medical device and the aspirator; and
placing another assembly of a chamber and a filter between the elongated medical device and the aspirator.

20. The method of claim 12, further comprising:
tilting or turning the chamber to defog the chamber.
